(19) United States
(12) Patent Application Publication
(10) Pub. No.: US 2016/0185632 A1
(43) Pub. Date: Jun. 30, 2016

(54) METHOD AND INSTALLATION FOR REMOVING SULPHUR FROM THE DIGESTATE AND THE BIOGAS OF A DIGESTER

(71) Applicant: R + I ALLIANCE, Paris La Defense Cedex (FR)

(72) Inventors: Yves LESTY, Las Condes (CL); Rolando CHAMY, Valparaiso (CL)

(21) Appl. No.: 14/898,349

(22) PCT Filed: Jun. 12, 2014
(86) PCT No.: PCT/IB2014/062171

§ 371 Date: Dec. 14, 2015

(30) Foreign Application Priority Data
Jun. 14, 2013 (FR) FR 1355538

(51) Int. Cl. C02F 1/74; C12M 1/107; C02F 11/04; C12M 1/00; C02F 3/20; C02F 3/28

(52) U.S. Cl. CPC C02F 1/74; C02F 3/20; C02F 3/28; C02F 11/04; C12M 29/24; C12M 21/04; C12M 23/36; C02F 2101/101

(57) ABSTRACT

Method for removing sulphur from the digestate and the biogas in a city and/or agricultural and/or industrial effluent digester, using a wet and/or a dry route, the digester (1) being made up of a closed vessel (2) in which a mass of products to be treated, that forms the digestate, undergoes anaerobic digestion with a volume of gas on top of the digestate from which the biogas is extracted, the digestate being drawn off at a point (5) of the digester then reinjected at another point (7). The digestate drawn off is made to follow: an upward first path (10) with injection of gaseous oxidizing agent at the bottom for aeration, the gaseous oxidizing agent and the digestate flowing concurrentwise; and a downward second path (12) under anaerobic conditions before returning to the digester, the excess gaseous oxidizing agent being removed in the top part (20) of at least one of the paths.

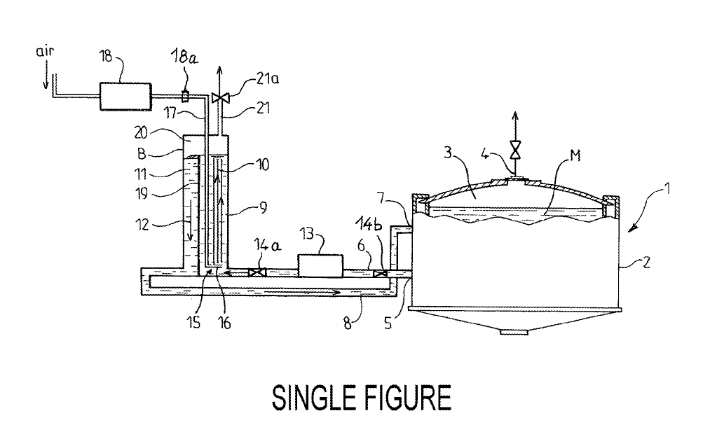

The invention relates to a method for total or partial desulfurization of the digestate and the biogas in a digester for municipal and/or agricultural and/or industrial effluents, by wet and/or dry route, the digester being made up of a closed vessel in which a mass of products for treatment is anaerobically digested, forming the digestate, the biogas being extracted from a gas space above the digestate, in which method the digestate is withdrawn at one point in the digester and then reinjected at another point.

A method of this generic type is known, in particular from document WO 2012/090139.

In the wastewater sector, the importance of sludge treatment is increasing, not only in terms of reducing waste but also to contribute to energy production by processes of anaerobic digestion.

The anaerobic digestion of sewage plant sludges produces significant amounts of biogas, which is a methane-rich gas that can be used for biofuel or energy production.

This biogas nevertheless contains some compounds which make it difficult to use, depending on the selected end use. These compounds include hydrogen sulfide, H₂S. This compound is harmful to human health and has a corrosive effect on apparatus. It is present at concentrations which can typically range between 0 and 10 000 ppm. This compound constitutes the primary target for removal from the biogas.

A variety of methods have been developed for maximum possible removal of hydrogen sulfide. Among these methods, those of the type to which the invention is directed prevent the accumulation of hydrogen sulfide in the biogas during the anaerobic digestion process.

According to the method of WO 2012/090139, gaseous or liquid oxidizing agent is injected into the digestate as it moves within a recirculation loop. Complete dissolution of the oxidizing agent, especially air or oxygen, in the digestate is not always satisfactorily obtained, and so a two-phase stream of digestate and oxidizing agent may be brought about, leading to reaction in the gas phase with deposition of sulfur on the walls of the digester.

The principal aim of the invention is to provide a desulfurization method of the generic type defined above that in the majority of cases results in complete dissolution of the oxidizing agent, more particularly of air or oxygen, in the digestate, and which permits a reduction in the frequency of maintenance operations on the digester, especially cleaning of its walls. It is desirable, moreover, that the method should be simple and economic to operate and should be able to be easily implemented on an existing digester.

According to the invention, a method for desulfurizing the digestate and the biogas, of the generic type defined above, is characterized in that the digestate withdrawn is subjected to:

an upward first path with injection of gaseous oxidizing agent at the bottom part for aeration, the gaseous oxidizing agent and the digestate circulating cocurrently,

and a downward second path, under anaerobic conditions, before return to the digester,

the excess gaseous oxidizing agent being evacuated in the top part of at least one of the paths.

The digestate is preferably contacted with the gaseous oxidizing agent in a separate device of the digester, for effective dissolution of the gaseous oxidizing agent.

When the digester comprises at least one external digestate recirculation loop, the upward first path and the downward second path for the digestate are advantageously provided in the recirculation loop.

The upward first path is preferably effected in an aeration chamber extending essentially vertically, while the downward second path is effected in a deaeration chamber extending essentially vertically.

The passage from the end of the upward first path to the start of the downward second path may be ensured by pouring the digestate over the upper edge of a partition.

The gaseous oxidizing agent may be air.

The gaseous oxidizing agent is advantageously injected at the bottom part of the aeration chamber, with effluent for treatment likewise entering at the bottom part of the aeration chamber.

The invention also relates to a biogas production plant comprising a digester for municipal and/or agricultural and/or industrial effluents, by wet and/or dry route, the digester being made up of a closed vessel in which a mass of products for treatment is anaerobically digested, forming a digestate, the biogas being extracted from a gas space above the digestate, said plant including, on the digester, a point for withdrawal of digestate and a point for reinjection into the digester, this plant being characterized in that it comprises:

a means defining an upward first path of the digestate withdrawn, with a means for injection of gaseous oxidizing agent at the bottom part of the path, for aeration of the digestate;

a second means, defining a downward second path, under anaerobic conditions, before return to the digester;

and a means for evacuating excess gaseous oxidizing agent in the top part of at least one of the paths.

The means defining the upward first path preferably consists of an aeration chamber extending essentially vertically, while the second means, defining the downward second path, consists of a deaeration chamber extending essentially vertically.

The aeration chamber and the deaeration chamber are advantageously situated in a single vessel, particularly a prism-shaped vessel, which is disposed vertically and is divided internally into two spaces, separated by a partition, corresponding to the aeration and deaeration chambers. The height of the partition is preferably less than that of the vessel, and the deaeration chamber is supplied with effluent by pouring, from the aeration chamber, over the upper edge of the partition.

A vertical tube may be submerged in the aeration chamber for injection of the gaseous oxidizing agent at the bottom part of this chamber, the vertical tube being supplied with gaseous oxidizing agent under pressure from a compressor whose outlet is connected to the tube, it being possible for said tube to comprise, at its lower end, a diffuser which promotes the dispersion of the gaseous oxidizing agent in the digestate.

When the plant comprises at least one external digestate recirculation loop, the aeration chamber and the deaeration chamber are advantageously provided in the recirculation loop.

The means for evacuation advantageously consists of a vent in the top part of the aeration and deaeration chambers, and the methane content of the biogas is enriched through the entrainment of carbon dioxide greater than the entrainment of methane, in the vent gas above the aeration and deaeration chambers.

The nitrogen content of the biogas is reduced through the entrainment of nitrogen in the vent gas above the aeration and deaeration chambers.

Aside from the arrangements set out above, the invention constitutes a number of other arrangements which will be addressed more explicitly below, with reference to an exemplary embodiment which is described in relation to the attached drawing, but which has no limiting effect whatsoever.

The single FIGURE of the drawing is a scheme of a plant for implementing the method in accordance with the invention.

Before a description of the method and the plant in relation to the FIGURE of the drawing, a number of facts will be called with regard to anaerobic digestion. In digestion of this kind, the sulfates present in the sludge are reduced to sulfides by sulfate-reducing bacteria (SRB), and are subsequently transferred to the gas phase via the liquid/gas equilibrium. In another pathway, the anaerobic degradation of proteins also gives rise to sulfides.

Under conditions of limited oxygenation, and still without removing the digestion from its anaerobic conditions, it is possible for these sulfides to be oxidized to sulfur, instead of the sulfates, by another type of bacteria: sulfide-oxidizing bacteria (SOB).

The invention constitutes the development of a system or method which allows small amounts of oxygen to be dissolved in the sludge or digestate, promoting the direct conversion reaction of sulfides to elemental sulfur in the digestate phase (also called sludge phase), and by wholly or partly preventing the release of hydrogen sulfide H₂S into the gas. The invention allows the gaseous oxidizing agent to be dissolved in the sludge, avoiding a two-phase stream of sludge/digestate and gaseous oxidizing agent, which can lead to reaction in the gas phase of the digester.

According to the invention, gaseous oxidizing agent is injected such that the digestate or sludge is contacted with the gaseous oxidizing agent, generally air, in a separate device of the digester, especially an aeration chamber and a deaeration chamber, as explained hereinafter.

Referring to the drawing, a plant can be seen that comprises a digester 1 consisting of a closed vessel 2 in which a mass M of products for treatment is anaerobically digested, forming a digestate, the biogas being extracted via a line 4 from a gas space 3 above the digestate.

In the drawing, the proportions between the dimensions of the digester and those of the aeration and deaeration chambers addressed later on below are not the actual proportions.

The plant comprises, on the digester, a digestate withdrawal point 5, generally at the bottom part of the digester, to which a line 6 is connected. The digestate is returned at a reinjection point 7, generally situated at a higher level than that of the withdrawal point 5. A reinjection line 8 is connected to the point 7. A loop for recirculation of the digestate is formed accordingly by lines 6 and 8 collectively.

Installed within this recirculation loop there is an aeration chamber 9, which extends essentially vertically and constitutes a means for defining an upward first path 10 of the digestate withdrawn.

A deaeration chamber 11 follows the chamber 9, and constitutes a means defining a downward second path 12, under anaerobic conditions, before return of the digestate to the digester via the line 8, which is connected to the lower part of the chamber 11.

The digestate withdrawn is moved within lines 6 and 8 by a pump 13, especially a peristaltic pump, which is installed in the line 6. Valves 14a and 14b, which are disposed in the line 6, allow the pump 13 to be isolated. The flow rate of the withdrawn digestate can be adjusted by altering the rotary speed of the pump 13.

A means 15 for injecting gaseous oxidizing agent, preferably air, is provided at the bottom part of the upward path 10, in other words at the bottom part of the aeration chamber 9. This injection means 15 consists advantageously of a diffuser 16, sited at the lower end of a vertical tube 17 which is submerged in the digestate in the chamber 9. The supply of compressed air is provided by a compressor 18 whose outlet is connected to the tube 17. A flow meter 18a allows the flow rate of air introduced to be monitored.

The aeration chamber 9 and the deaeration chamber 11 are preferably situated in a single vessel B, in particular a prism-shaped vessel, whose generatrices are disposed vertically. The inner volume of the vessel B is divided into two spaces, which are separated by a partition 19 with a height less than that of the vessel B, so that a digestate-free space 20 is located at the top part of the vessel B. The deaeration chamber 11 is supplied with digestate by pouring the digestate, coming from the aeration chamber 9, over the upper edge of the partition 19.

A vent tube 21 opens into the upper part of the space 20 for the evacuation of the excess air which has not dissolved in the digestate during the upward path 10. This vent 21 constitutes a means for evacuation of the excess gaseous oxidizing agent in the top part of the aeration path 10, which also corresponds to the top part of the downward deaeration path 11. The diffuser 16 for injection of air/oxygen at the lower part of the aeration chamber 9 promotes the dissolution of the gaseous oxidizing agent in the digestate, which takes place during the cocurrent circulation of the digestate and the air, following the upward path 10. A non-return valve 21a is installed in the vent tube 21 to allow the evacuation of a vent gas, formed primarily of excess air and other gases, particularly carbon dioxide and methane, that are entrained. More carbon dioxide, CO₂, is entrained in the vent gas than is methane, and the biogas obtained from the digester will therefore be methane-enriched.

The aerated digestate is poured out at the top part of the aeration chamber 9, and falls by gravity into the deaeration chamber 11, which constitutes an anaerobic zone in which there is no addition of air/oxygen. Any remaining bubbles of air/oxygen are able to escape from the digestate via the upper free space 20, thereby preventing entry of excessive amounts of air/oxygen into the digester 1. Line 8 provides for the return of the aerated digestate to the digester.

The hydrogen sulfide which was present in the digestate withdrawn at point 5 has been converted, by the injection of oxidizing agent, into sulfur, which remains in the digestate or which is deposited on the walls of the deaeration chamber 11; consequently, hydrogen sulfide is removed from the biogas, wholly or partly, and the risks of deposits on the walls of the digester 1 are either limited or eliminated.

Although not vital, it is advantageous to implement the solution of the invention in digesters which use a digestate recirculation system for purposes of heating and/or mixing. In that case, the digestate recirculation stream is directed to the aeration chamber and the deaeration chamber. The inclusion of these chambers within the recirculation loop is sufficient.

A substantial advantage of the proposed plant with aeration and deaeration chambers is that the formation of sulfur inside the digester is prevented. This is optimum from the functional standpoint, since uncontrolled formation of sulfur on the walls and in the vessel of the digester can lead to unwanted halts in operation, to a decrease in anaerobic digestion performance and to a decrease in the availability of the plant. These treatment methods have a relatively long start-up period, of up to twenty days. Moreover, a reduced amount of nitrogen is found in the biogas, since some of the nitrogen has been removed by the vent above the aeration and deaeration chambers.

Trials were conducted in a pilot plant comprising two twin digesters each equipped with an aeration chamber 9 and a deaeration chamber 11. One of these digesters operated without air injection, while the other digester operated with injection of air into the aeration chamber in order to produce a micro-aeration system.

The comparative results confirmed the advantages of the invention in respect of:

the performance of the proposed device for micro-aeration,

the performance of the anaerobic digestion under the effects of the micro-aeration,

the optimization directives of the method,

a procedure for increasing the yield.

The method of the invention produces a micro-aeration system which is able wholly or partly to prevent the formation of hydrogen sulfide in any environment of biogas production by anaerobic digestion.

As an example, a reduction was obtained of 49.8% of hydrogen sulfide in the biogas, for a micro-aeration of 0.394 Sm³ of air per m³ of digester per day; the methane concentration in the biogas was 4% to 5% greater in the micro-aereated digester, while the carbon dioxide was proportionally lower; the concentration of nitrogen in the biogas from this digester still remained less than 3%.

The invention is applicable to any industrial environment which involves production of biogas, and particularly wastewater treatment stations, discharges, digestion of agricultural products for energy production, and for which the hydrogen sulfide H₂S in the biogas represents a target pollutant which must be removed or whose formation must be prevented. Where appropriate, other advantages associated with the reduction in costs and with environmental compliance may result from the invention:

if ferric chloride, FeCl₃, is used to minimize the production of hydrogen sulfide H₂S, this expensive chemical consumption can be reduced or avoided;

there may be reduced consumption of chemical cleaners and of absorption materials, such as of activated carbon, for example;

depending on the end use and the concentration of hydrogen sulfide H₂S obtained, there might be complete avoidance of any capital investment for the treatment of the biogas;

for an end use which is tolerant of yet sensitive to hydrogen sulfide, an increase in lifetime is ensured -for example, for an internal combustion engine;

the emission of oxides of sulfur, originating from any combustion in an end use, is reduced or avoided.

More specifically, the micro-aeration method according to the invention provides all of the benefits of a conventional micro-aeration, while preventing clogging by sulfur within the digester, thereby diminishing the problems of operation and of falls in yield of the anaerobic digestion. Other noteworthy points include the increase in the methane content of the biogas and the reduction in the carbon dioxide and nitrogen contents.

All of these results may be obtained with a minimum of capital investment, since provision of an aeration chamber and a deaeration chamber is sufficient.

Implementation is even more direct in the case of digesters which utilize recirculation of digestate as a means of mixing, since in that case the aeration and deaeration chambers are installed in the loop.

1. A method for desulfurizing the digestate and the biogas in a digester for municipal and/or agricultural and/or industrial effluents, by wet and/or dry route, the digester being made up of a closed vessel in which a mass of products for treatment is anaerobically digested, forming the digestate, the biogas being extracted from a gas space above the digestate, in which method the digestate is withdrawn at one point in the digester and then reinjected at another point, wherein the digestate withdrawn is subjected to:
an upward first path with injection of gaseous oxidizing agent at the bottom part for aeration, the gaseous oxidizing agent and the digestate circulating cocurrently,
and a downward second path, under anaerobic conditions, before return to the digester,
the excess gaseous oxidizing agent being evacuated in the top part of at least one of the paths.

2. The method as claimed in claim 1, wherein the digestate is contacted with the gaseous oxidizing agent in a separate device of the digester, for effective dissolution of the gaseous oxidizing agent.

3. The method as claimed in claim 1, for a digester comprising at least one external digestate recirculation loop, wherein the upward first path and the downward second path for the digestate are provided in the recirculation loop.

4. The method as claimed in claim 1, wherein the upward first path is effected in an aeration chamber extending essentially vertically, while the downward second path is effected in a deaeration chamber extending essentially vertically.

5. The method as claimed in claim 4, wherein passage from the end of the upward first path to the start of the downward second path is ensured by pouring the digestate over the upper edge of a partition.

6. The method as claimed in claim 1, wherein the gaseous oxidizing agent is air.

7. The method as claimed in claim 4, wherein the gaseous oxidizing agent is injected at the bottom part of the aeration chamber, with effluent for treatment likewise entering at the bottom part of the aeration chamber.

8. A biogas production plant comprising a digester for municipal and/or agricultural and/or industrial effluents, by wet and/or dry route, the digester being made up of a closed vessel in which a mass of products for treatment is anaerobically digested, forming a digestate, the biogas being extracted from a gas space above the digestate, said plant including, on the digester, a point for withdrawal of digestate and a point for reinjection into the digester, this plant being further comprising:
a means defining an upward first path of the digestate withdrawn, with a means for injection of gaseous oxidizing agent at the bottom part of the path, for aeration of the digestate;
a second means, defining a downward second path, under anaerobic conditions, before return to the digester;
and a means for evacuating excess gaseous oxidizing agent in the top part of at least one of the paths.

9. The plant as claimed in claim 8, wherein the means defining the upward first path comprises an aeration chamber extending essentially vertically, while the second means, defining the downward second path, comprises a deaeration chamber extending essentially vertically.

10. The plant as claimed in claim 9, wherein the aeration chamber and the deaeration chamber are situated in a single vessel, particularly a prism-shaped vessel, which is disposed vertically and is divided internally into two spaces, separated by a partition, corresponding to the aeration and deaeration chambers.

11. The plant as claimed in claim 10, wherein the height of the partition is less than that of the vessel, and the deaeration chamber is supplied with effluent by pouring, from the aeration chamber, over the upper edge of the partition.

12. The plant as claimed in claim 10, wherein a vertical tube is submerged in the aeration chamber for injection of the gaseous oxidizing agent at the bottom part of this chamber, the vertical tube being supplied with gaseous oxidizing agent under pressure from a compressor whose outlet is connected to the tube.

13. The plant as claimed in claim 12, wherein the tube for injection of the gaseous oxidizing agent comprises at its lower end a diffuser which promotes the dispersion of the gaseous oxidizing agent in the digestate.

14. The plant as claimed in claim 9, comprising at least one external digestate recirculation loop, wherein the aeration chamber and the deaeration chamber are provided in the recirculation loop.

15. The plant as claimed in claim 9, wherein the means for evacuation comprises a vent in the top part of the aeration and deaeration chambers, and the methane content of the biogas is enriched through the entrainment of carbon dioxide greater than the entrainment of methane, in the vent gas above the aeration and deaeration chambers.

16. The plant as claimed in claim 15, wherein the nitrogen content of the biogas is reduced through the entrainment of nitrogen in the vent gas above the aeration and deaeration chambers.
